(19) United States
(12) Patent Application Publication
(10) Pub. No.: US 2026/0248569 A1
(43) Pub. Date: Aug. 27, 2026

(54) ARTIFICIAL MUSCLE-DRIVEN HYBRID FRACTURE REDUCTION SURGICAL ROBOT

(71) Applicant: Harbin Institute of Technology, Harbin (CN)

(72) Inventors: Hongjian YU, Harbin (CN); Xiangyu SHEN, Harbin (CN); Zijia GUO, Harbin (CN); Dongru XIE, Harbin (CN); Mingxuan CHEN, Harbin (CN)

(21) Appl. No.: 19/641,631

(22) Filed: Apr. 8, 2026

(30) Foreign Application Priority Data
Apr. 14, 2025 (CN) 202510467498.0

(51) Int. Cl. A61B 34/30

(52) U.S. Cl. CPC A61B 34/30; A61B 2034/304; A61B 2034/306

(57) ABSTRACT

An artificial muscle-driven hybrid fracture reduction surgical robot is disclosed, and pertains to the technical field of auxiliary operating instruments for medical surgery. In the present disclosure, the problems associated with existing fracture reduction surgical robots, such as their large size, limited workspace, low integration level, insufficient load capacity, complex control and kinematics, and being prone to cause rigid impacts during motion are addressed. In the present invention, a top side of a mounting base is provided with a dual-joint swing arm, and a first rotary joint is mounted at a first end of a swing arm connecting rod. Left and right ends of the first rotary joint are connected to two first rotary joint rotation and traction mechanisms, respectively, a bottom of the first rotary joint is connected to the mounting base, a second end of the swing arm connecting rod is mounted with a second rotary joint.

### TECHNICAL FIELD

The present disclosure pertains to the technical field of auxiliary operating instruments for medical surgery, and particularly to an artificial muscle-driven hybrid fracture reduction surgical robot.

### BACKGROUND

As one of the most frequent clinical conditions in orthopedics, fractures pose a significant threat to patient health. Among these, pelvic fractures, constituting approximately 1%-3% of total fracture cases, are associated with disability rates of 50%-60% and mortality rates exceeding 13%. Fracture reduction represents the most critical step in orthopedic surgery. A reduction procedure characterized by minimal trauma, high precision, and high reliability is essential for both surgical success and postoperative patient recovery. Currently, conventional clinical methods for fracture reduction primarily fall into two categories: manual reduction with external splint fixation and open reduction with internal plate fixation. The principal advantage of manual reduction lies in its avoidance of an incision at the fracture site, thereby reducing the risk of infection. Nevertheless, due to the inability to obtain fracture information intuitively, this technique imposes stringent requirements on the operating physician and frequently necessitates multiple reduction maneuvers, prone to causing secondary fractures. In contrast, the advantage of open reduction is that physicians can directly acquire fracture information through visual or tactile means, thereby reducing the number of reductions and improving reduction accuracy. Nevertheless, it also significantly increases the potential for surgical site infection. The use of a fracture reduction robotic system in fracture surgery, integrating high-precision mechanical components and servo control components with medical image-based planning and navigation technologies, enables the robot to perform the reduction operation autonomously. This method can significantly improve the accuracy and reliability of reduction while mitigating infection risk, preserving the operating physician's stamina, decreasing radiation exposure duration, and promoting the dissemination of technically demanding fracture procedures.

Fracture reduction surgical robots are primarily configured in three architectures: serial, parallel, and serial-parallel hybrid connections. Conventional parallel robots offer advantages such as structural stability, high rigidity, and high precision. However, they are typically limited by a small workspace, complex control requirements, and complicated kinematic solutions. Although serial robots provide a large workspace and high flexibility, they also occupy significant surgical space, which can restrict the operating area for medical staff. Furthermore, the cumulative error inherent in serial mechanisms leads to reduced positioning accuracy of the robotic end-effectors. A hybrid robot system can integrate the advantages of both serial and parallel configurations to a certain extent. Regarding the actuation method, existing fracture reduction robots are predominantly driven by motors. Motor drives offer high precision, fast response, convenient speed regulation, and minimal contamination. A limitation of the motor drivers is the relatively small thrust output. To improve output torque, they typically require the addition of a corresponding reducer. Artificial muscles, composed of flexible and stretchable materials, can maintain excellent compliance in complex environments and avoid generating rigid impacts during motion. Simultaneously, compared to conventional rigid actuators, artificial muscles exhibit higher efficiency in energy conversion and transmission, enabling more effective energy utilization.

### SUMMARY

An objective of the present disclosure is to address the problems associated with existing fracture reduction surgical robots, such as their large size, limited workspace, low integration level, insufficient load capacity, complex control and kinematics, and being prone to causing rigid impacts during motion. Accordingly, the present disclosure provides an artificial muscle-driven hybrid fracture reduction surgical robot.

The technical solution of the present disclosure is:
an artificial muscle-driven hybrid fracture reduction surgical robot, which includes a dual-joint swing arm 1, a four-limb two rotations and one translation (2R1T) parallel mechanism 2, an end traction platform 3 and a robot base 4, wherein the robot base 4 includes a mounting base 41, two first rotary joint rotation and traction mechanisms 42 and two second rotary joint rotation and traction mechanisms 43, the mounting base 41 is vertically arranged. The first rotary joint rotation and traction mechanism 42 and the second rotary joint rotation and traction mechanism 43 are arranged on left and/or right sides of the mounting base 41. A top side of the mounting base 41 is provided with the dual-joint swing arm 1. The dual-joint swing arm 1 includes a first rotary joint 11, a swing arm connecting rod 12 and a second rotary joint 13. The swing arm connecting rod 12 is arranged horizontally. The first rotary joint 11 is mounted at a first end of the swing arm connecting rod 12. Left and right ends of the first rotary joint 11 are connected to two first rotary joint rotation and traction mechanisms 42, respectively, a bottom of the first rotary joint 11 is connected to the mounting base 41, a second end of the swing arm connecting rod 12 is mounted with the second rotary joint 13, left and right ends of the second rotary joint 13 are connected to two second rotary joint rotation and traction mechanisms 43, respectively, a top of the second rotary joint 13 is connected to the vertically arranged four-limb 2R1T parallel mechanism 2, and a top end of the four-limb 2R1T parallel mechanism 2 is mounted with an end traction platform 3.

Further, the first rotary joint 11 includes a first rotary joint bearing 111, a first rotary joint bearing housing 112 and a base adapter module 113, a first rotary joint mounting hole vertically penetrating upper and lower surfaces of the swing arm connecting rod 12 is machined at a first end of the swing arm connecting rod 12, the first rotary joint bearing 111 is embedded inside the first rotary joint mounting hole, an upper end of the first rotary joint bearing housing 112 is inserted into an inner hole of the first rotary joint bearing 111, and a lower end of the first rotary joint bearing housing 112 is connected to the base adapter module 113.

Further, the second rotary joint 13 includes a second rotary joint bearing, a second rotary joint bearing housing 131 and a 2R1T adapter module 132, a second rotary joint mounting hole vertically penetrating upper and lower surfaces of the swing arm connecting rod 12 is machined at a second end of the swing arm connecting rod 12, the second rotary joint bearing is embedded inside the second rotary joint mounting hole, a lower end of the second rotary joint bearing housing 131 is inserted into an inner hole of the second rotary joint bearing, and an upper end of the second rotary joint bearing housing 131 is connected to the 2R1T adapter module 132.

Further, one end of the swing arm connecting rod 12 adjacent to the robot base 4 is machined into a circular arc surface, and the circular arc surface is machined with two first horizontal rope grooves arranged side by side in an upper-lower arrangement, and two rope holes penetrating an inner side surface of the first rotary joint mounting hole are machined at a first end of the swing arm connecting rod 12, and the two rope holes correspond to the two first horizontal rope grooves one-to-one respectively;
the first rotary joint rotation and traction mechanism 42 includes two first pneumatic artificial muscles 421, two first rotary joint rotary traction ropes 422, two first rotary joint guide wheel mounting brackets 423 and four first rotary joint guide wheels 424, the two first pneumatic artificial muscles 421 are vertically arranged on left and right sides of the mounting base 41, respectively. The first rotary joint guide wheel mounting bracket 423 is arranged directly above each first pneumatic artificial muscle 421, the first rotary joint guide wheel mounting bracket 423 is configured as an L-shaped block structure, an inner right-angled corner of the first rotary joint guide wheel mounting bracket 423 is mounted at a front right-angled corner of the mounting base 41, the vertically arranged first rotary joint guide wheel 424 is mounted on an outer end surface of the first rotary joint guide wheel mounting bracket 423, an outer right-angled corner of the first rotary joint guide wheel mounting bracket 423 is provided with a horizontally arranged first outer edge, a lower part of the first outer edge is mounted with the horizontally arranged first rotary joint guide wheel 424, a free end of the first pneumatic artificial muscle 421 is connected to a first end of the first rotary joint rotary traction rope 422, a second end of the first rotary joint rotary traction rope 422 sequentially passes around the vertically arranged first rotary joint guide wheel 424, the horizontally arranged first rotary joint guide wheel 424, the first horizontal rope groove and the rope hole at the first end of the swing arm connecting rod 12, and finally connects to a left end and/or a right end of the first rotary joint bearing housing 112.

Further, a side surface of a second end of the second rotary joint bearing housing 131 is machined into a circular arc surface, and the circular arc surface of the second end of the second rotary joint bearing housing 131 is machined with two second horizontal rope grooves arranged side by side in an upper-lower arrangement;
the second rotary joint rotation and traction mechanism 43 includes two second pneumatic artificial muscles 431, two second rotary joint rotary traction ropes 432, two second rotary joint guide wheel mounting brackets 433 and four second rotary joint guide wheels 434, the two second pneumatic artificial muscles 431 are vertically arranged on left and right sides of the mounting base 41, respectively, the second rotary joint guide wheel mounting bracket 433 is arranged directly above each second pneumatic artificial muscle 431, the second rotary joint guide wheel mounting bracket 433 is configured as an L-shaped block structure, an inner right-angled corner of the second rotary joint guide wheel mounting bracket 433 is mounted at a front right-angled corner of the mounting base 41, the vertically arranged second rotary joint guide wheel 434 is mounted on an outer end surface of the second rotary joint guide wheel mounting bracket 433, an outer right-angled corner of the second rotary joint guide wheel mounting bracket 433 is provided with a horizontally arranged second outer edge, a lower part of the second outer edge is mounted with the horizontally arranged second rotary joint guide wheel 434, a free end of the second pneumatic artificial muscle 431 is connected to a first end of the second rotary joint rotary traction rope 432, a second end of the second rotary joint rotary traction rope 432 sequentially passes around the vertically arranged second rotary joint guide wheel 434, the horizontally arranged second rotary joint guide wheel 434, the second horizontal rope groove at a second end of the second rotary joint bearing housing 131, and finally connects to a left end and/or a right end of the second rotary joint bearing housing 131.

Further, the robot base 4 further includes two base artificial muscle mounting brackets 44, the base artificial muscle mounting bracket 44 is a block structure with a right triangle cross section, the two base artificial muscle mounting brackets 44 are horizontally arranged on left and right sides of the mounting base 41 and arranged above the first pneumatic artificial muscle 421 and the second pneumatic artificial muscle 431, respectively, an inner vertical surface of the base artificial muscle mounting bracket 44 is fixedly connected to a side of the mounting base 41, and an upper horizontal surface of the base artificial muscle mounting bracket 44 is fixedly connected to lower surfaces of the first pneumatic artificial muscle 421 and the second pneumatic artificial muscle 431.

Further, the four-limb 2R1T parallel mechanism 2 includes a parallel mechanism base 21, two ascending limbs 22, two descending limbs 23, four optical axes 24, four universal joint (UU) mechanisms 25 and eight linear bearings 26; the parallel mechanism base 21 includes a mounting plate connecting column 211, an adapter module connecting plate 212 and two mounting plates 213. The mounting plate connecting column 211 is vertically arranged. Two mounting plates 213 are arranged horizontally on upper and lower sides of the mounting plate connecting column 211. Each mounting plate 213 is machined with four mounting plate mounting holes that penetrate upper and lower surfaces of the mounting plate and are arranged in a rectangular array. Lower ends of the four optical axes 24 sequentially pass through the mounting plate mounting holes of the two mounting plates 213. The linear shaft 24 is connected to the mounting plate mounting holes through the linear bearing 26 in a sliding manner. Upper ends of the four optical axes 24 are connected to lower ends of the four UU mechanisms 25, respectively. The upper mounting plate 213 is provided with the adapter module connecting plate 212 adjacent to a side of the dual-joint swing arm 1. The adapter module connecting plate 212 is located above the 2R1T adapter module 132, and the adapter module connecting plate 212 is detachably connected to the 2R1T adapter module 132 through multiple connectors. The upper mounting plate 213 is machined with two ascending limb mounting holes symmetrically arranged on left and right sides centered on an axis of the parallel mechanism base 21. Second ends of the two ascending limbs 22 are vertically inserted into the two ascending limb mounting holes, respectively, and first ends of the two ascending limbs 22 are connected to upper parts of the linear shaft 24 at two diagonal positions, respectively. The lower mounting plate 213 is machined with two descending limb mounting holes arranged in front and rear positions centered on an axis of the parallel mechanism base 21. Second ends of the descending limbs 23 are vertically inserted into the two descending limb mounting holes, and first ends of the descending limbs 23 are connected to lower parts of the linear shaft 24 at remaining two diagonals, respectively.

Further, each ascending limb 22 includes a lifting pneumatic artificial muscle 221 and a lifting artificial muscle free end locking part 222. Two ends of the lifting artificial muscle free end locking part 222 are machined with a lifting artificial muscle free end mounting hole and an upper mounting hole for the linear shaft that penetrate upper and lower surfaces of the locking part, respectively, a free end of the lifting pneumatic artificial muscle 221 is inserted in the lifting artificial muscle free end mounting hole, and a fixed end of the lifting pneumatic artificial muscle 221 is inserted in the ascending limb mounting hole of the parallel mechanism base 21, wherein the optical axes 24 at the two diagonals are inserted in the ascending limb mounting hole. Each descending limb 23 includes a pull-down pneumatic artificial muscle 231 and a pull-down artificial muscle free end locking part 232, both ends of the pull-down artificial muscle free end locking part 232 are machined with a pull-down artificial muscle free end mounting hole and a lower mounting hole for the linear shaft that penetrate the upper and lower surfaces of the locking part, respectively, a free end of the pull-down pneumatic artificial muscle 231 is inserted in the pull-down artificial muscle free end mounting hole, and a fixed end of the pull-down pneumatic artificial muscle 231 is inserted in the descending limb mounting hole of the parallel mechanism base 21, wherein the remaining two diagonal optical axes 24 are inserted in the descending limb mounting hole.

Further, each UU mechanism 25 includes a first connecting frame 251, a first connecting pin 252, a connecting rod 253, a second connecting pin 254 and a second connecting frame 255. The first connecting frame 251 and the second connecting frame 255 are all U-shaped structures. The U-shaped structure includes a U-shaped frame and a connecting shaft that is vertically arranged and integrated with a middle part of the U-shaped frame web. Two pin holes with a coaxial arrangement are arranged on the two wing plates of the U-shaped frame, and two connecting rod pin holes are arranged at both ends of the connecting rod 253. Both ends of the connecting rod 253 are rotatably connected to the U-shaped frames of the first connecting frame 251 and the second connecting frame 255 by the first connecting pin 252 and the second connecting pin 254, respectively, and a connecting shaft of the second connecting frame 255 is connected to the end traction platform 3.

Further, the end traction platform 3 includes a traction platform rigid rod member 31, a linear guide 36, two traction pneumatic artificial muscles 32, two wire ropes 33, two guide wheels 34, two guide wheel mounting brackets 35, two sliders 37 and two moving platforms 38, a bottom of the traction platform rigid rod member 31 is mounted with the linear guide 36 in a parallel arrangement, two sliders 37 are slidably mounted on the linear guide 36 in a parallel arrangement, and two symmetrically arranged moving platforms 38 are mounted on bottoms of the two sliders 37, end surfaces on both sides of the two moving platforms 38 are provided with two horizontal axis holes arranged obliquely and symmetrically, respectively. The axes of the two horizontal axis holes intersect with a middle line of the moving platform 38. Two traction pneumatic artificial muscles 32 are arranged on both sides of the traction platform rigid rod member 31 in a parallel arrangement. Two ear plates are arranged vertically at both ends of the traction platform rigid rod member 31, the two ear plates are located on both sides of the traction platform rigid rod member 31, fixed ends of the two traction pneumatic artificial muscles 32 are mounted on the corresponding side ear plates, respectively, free ends of the two traction pneumatic artificial muscles 32 are arranged inward, and a vertically arranged guide wheel is arranged directly in front of a free end of each traction pneumatic artificial muscle 32. The guide wheel 34 is mounted at an end of the traction platform rigid rod member 31 through the guide wheel mounting bracket 35, free ends of the two traction pneumatic artificial muscles 32 are connected to first ends of the two wire ropes 33, respectively, and second ends of the two wire ropes 33 pass around the two guide wheels 34 respectively, and connect to the two moving platforms 38. The four horizontal axis holes on the two moving platforms 38 are inserted with the connecting shaft of the second connecting frame 255 of the four UU mechanisms 25, respectively.

Compared with the prior art, the present disclosure has the following effects:
1. In order to address the challenges posed by the compact environment and confined space inherent in fracture reduction surgery, the present disclosure provides an artificial muscle-driven hybrid fracture reduction surgical robot, relative to existing fracture reduction robot designs, the proposed robot enhances the operational workspace by adjusting the configuration of its hybrid components under the precondition of ensuring a sufficiently large workspace. Meanwhile, it achieves an improvement in the system integration degree. Consequently, the structure of the robot becomes more compact, leading to a reduction in the space it occupies within the operating room.
2. The artificial muscle-driven hybrid fracture reduction surgical robot of the present disclosure features a relatively simple kinematic model in the robot kinematics solution, which is convenient for the kinematics solution.
3. In the artificial muscle-driven hybrid fracture reduction surgical robot, each joint is actuated by multiple artificial muscles configured in an antagonistic manner. This design enables the actuation of a redundant mechanism, thereby simplifying the complexity of the drive mechanism and further contributing to a reduction in overall volume.
4. The robot-assisted fracture reduction process involves the robot overcoming the traction resistance of human soft tissues and maneuvering the bone fragments to achieve reduction. During this process, inherent inaccuracies in modeling and the complex nature of soft tissues necessitate that the robot exhibit a degree of compliance. This compliance allows it to better adapt to the human soft tissues, thereby enhancing the safety of reduction. As a novel actuation element, artificial muscles possess compliance characteristics similar to human soft tissues while simultaneously providing high resetting force. Consequently, compared to existing electrically driven reduction robots, the artificial muscle-driven hybrid fracture reduction surgical robot of the present disclosure improves reduction flexibility and load capacity of the reduction robot by adopting artificial muscle actuation.

### BRIEF DESCRIPTION OF THE DRAWINGS

FIG. 1 is a schematic structural diagram of an artificial muscle-driven hybrid fracture reduction surgical robot according to the present disclosure;

FIG. 2 is a schematic structural diagram of a four-limb 2R1T parallel mechanism 2 after a UU mechanism 25 is removed according to the present disclosure;

FIG. 3 is a schematic structural diagram of an end traction platform 3 according to the present disclosure;

FIG. 4 is a schematic diagram of a connection between a linear shaft 24 and a UU mechanism 25 in a four-limb 2R1T parallel mechanism 2 according to the present disclosure;

FIG. 5 is a schematic structural diagram of a dual-joint swing arm 1 and a robot base 4 when they are combined according to the present disclosure.

Reference numerals in figures: 1, a dual-joint swing arm; 11, a first rotary joint; 111, a first rotary joint bearing; 112, a first rotary joint bearing housing; 113, a base adapter module; 12, a swing arm connecting rod; 13, a second rotary joint; 131, a second rotary joint bearing housing; 132, a 2R1T adapter module; 2, a four-limb 2R1T parallel mechanism; 21, a parallel mechanism base; 211, a mounting plate connecting column; 212, an adapter module connecting plate; 213, a mounting plate; 22, an ascending limb; 221, a lifting pneumatic artificial muscle; 222, a lifting artificial muscle free end locking part; 23, descending limb; 231, a pull-down pneumatic artificial muscle; 232, a pull-down artificial muscle free end locking part; 24, a linear shaft; 25, a UU mechanism; 251, a first connecting frame; 252, a first connecting pin; 253, a connecting rod; 254, a second connecting pin; 255, a second connecting frame; 26, a linear bearing; 3, an end traction platform; 31, a traction platform rigid rod member; 32, a traction pneumatic artificial muscle; 33, a wire rope; 34, a guide wheel; 35, a guide wheel mounting bracket; 36, a linear guide; 37, a slider; 38, a moving platform; 4, a robot base; 41, a mounting base; 42, a first rotary joint rotation and traction mechanism; 421, a first pneumatic artificial muscle; 422, a first rotary joint rotary traction rope; 423, a first rotary joint guide wheel mounting bracket; 424, a first rotary joint guide wheel; 43, a second rotary joint rotation and traction mechanism; 431, a second pneumatic artificial muscle; 432, a second rotary joint rotary traction rope; 433, a second rotary joint guide wheel mounting bracket; 434, a second rotary joint guide wheel; 44, a base artificial muscle mounting bracket.

### DETAILED DESCRIPTION

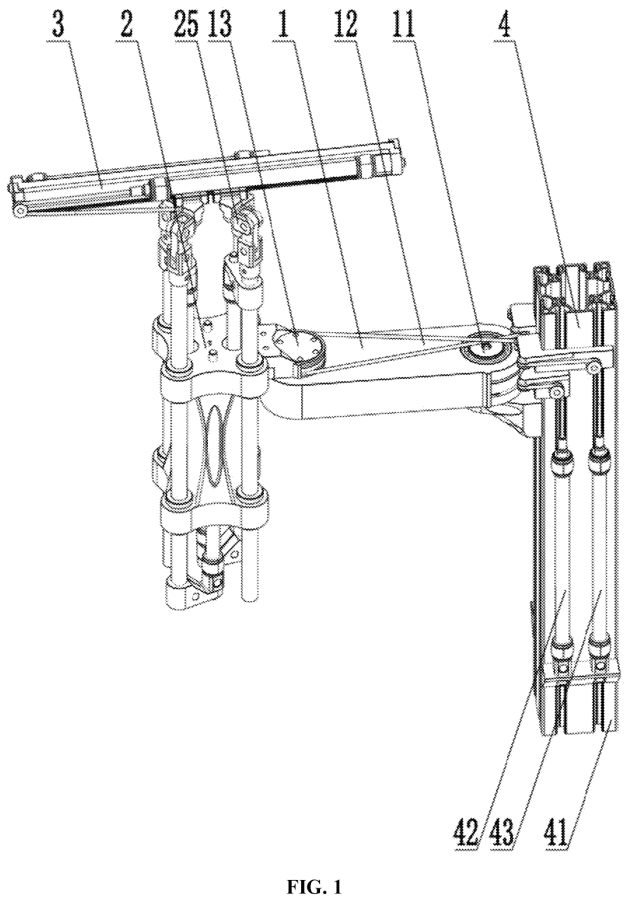

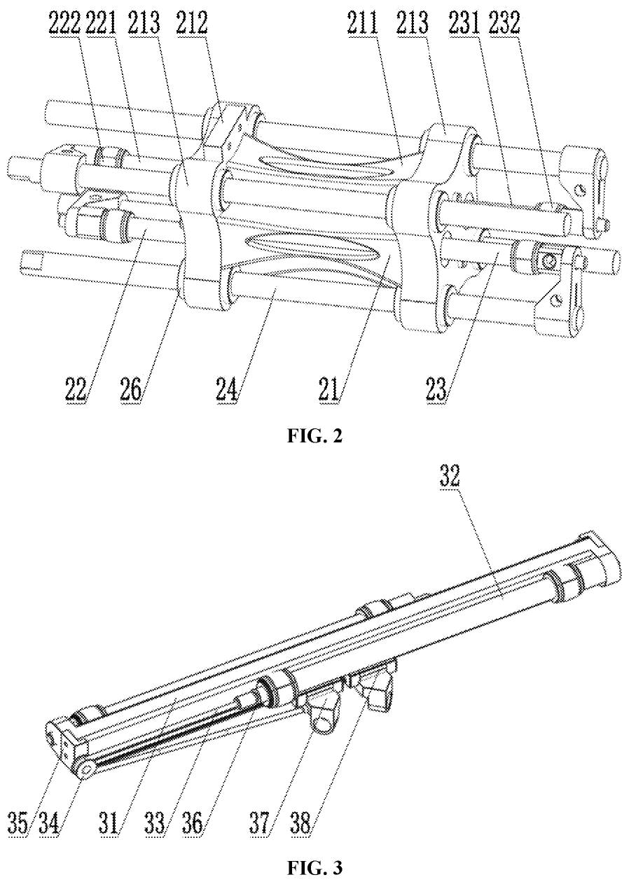

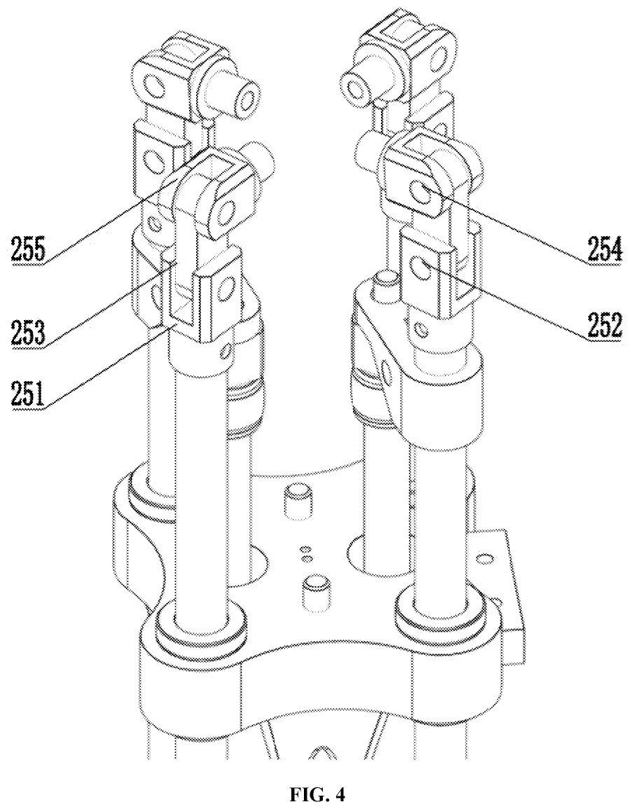

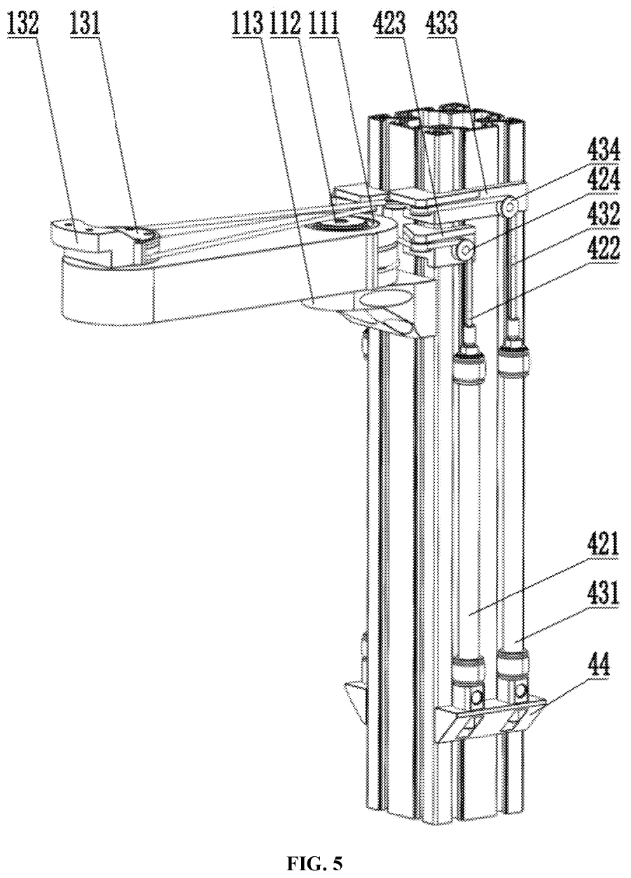

Detailed description of Embodiment 1: the embodiment is described with reference to FIGS. 1 to 5, according to the present embodiment, an artificial muscle-driven hybrid fracture reduction surgical robot includes the dual-joint swing arm 1, the four-limb 2R1T parallel mechanism 2, the end traction platform 3 and the robot base 4, wherein the robot base 4 includes the mounting base 41, two first rotary joint rotation and traction mechanisms 42 and two second rotary joint rotation and traction mechanisms 43, the mounting base 41 is vertically arranged. The first rotary joint rotation and traction mechanism 42 and the second rotary joint rotation and traction mechanism 43 are arranged on left and/or right sides of the mounting base 41. the top side of the mounting base 41 is provided with the dual-joint swing arm 1. The dual-joint swing arm 1 includes the first rotary joint 11, the swing arm connecting rod 12 and the second rotary joint 13. The swing arm connecting rod 12 is arranged horizontally. The first rotary joint 11 is mounted at the first end of the swing arm connecting rod 12. Left and right ends of the first rotary joint 11 are connected to two first rotary joint rotation and traction mechanisms 42, respectively, the bottom of the first rotary joint 11 is connected to the mounting base 41, the second end of the swing arm connecting rod 12 is mounted with the second rotary joint 13, left and right ends of the second rotary joint 13 are connected to two second rotary joint rotation and traction mechanisms 43, respectively, the top of the second rotary joint 13 is connected to the vertically arranged four-limb 2R1T parallel mechanism 2, and the top end of the four-limb 2R1T parallel mechanism 2 is mounted with the end traction platform 3.

Specifically, the swing arm connecting rod 12 is a rigid rod member. The mounting base 41 is a rod-shaped structure having a square cross-section.

Detailed description of Embodiment 2: the embodiment is described with reference to FIGS. 1 to 5, according to the present embodiment, the first rotary joint 11 includes the first rotary joint bearing 111, the first rotary joint bearing housing 112 and the base adapter module 113, the first rotary joint mounting hole vertically penetrating upper and lower surfaces of the swing arm connecting rod 12 is machined at the first end of the swing arm connecting rod 12, the first rotary joint bearing 111 is embedded inside the first rotary joint mounting hole, the upper end of the first rotary joint bearing housing 112 is inserted into the inner hole of the first rotary joint bearing 111, and the lower end of the first rotary joint bearing housing 112 is connected to the base adapter module 113. The other components and connection relationships are identical to those in Embodiment 1.

Detailed description of Embodiment 3: the embodiment is described with reference to FIGS. 1 to 5, according to the present embodiment, the second rotary joint 13 includes the second rotary joint bearing, the second rotary joint bearing housing 131 and the 2R1T adapter module 132, the second rotary joint mounting hole vertically penetrating upper and lower surfaces of the swing arm connecting rod 12 is machined at the second end of the swing arm connecting rod 12, the second rotary joint bearing is embedded inside the second rotary joint mounting hole, the lower end of the second rotary joint bearing housing 131 is inserted into the inner hole of the second rotary joint bearing, and the upper end of the second rotary joint bearing housing 131 is connected to the 2R1T adapter module 132. The other components and connection relationships are identical to those in Embodiment 1 or Embodiment 2.

Detailed description of Embodiment 4: the embodiment is described with reference to FIGS. 1 to 5, according to the present embodiment, one end of the swing arm connecting rod 12 adjacent to the robot base 4 is machined into the circular arc surface, and the circular arc surface is machined with two first horizontal rope grooves arranged side by side in the upper-lower arrangement, and two rope holes penetrating the inner side surface of the first rotary joint mounting hole are machined at the first end of the swing arm connecting rod 12, and the two rope holes correspond to the two first horizontal rope grooves one-to-one respectively;
the first rotary joint rotation and traction mechanism 42 includes two first pneumatic artificial muscles 421, two first rotary joint rotary traction ropes 422, two first rotary joint guide wheel mounting brackets 423 and four first rotary joint guide wheels 424, the two first pneumatic artificial muscles 421 are vertically arranged on left and right sides of the mounting base 41, respectively, the first rotary joint guide wheel mounting bracket 423 is arranged directly above each first pneumatic artificial muscle 421, the first rotary joint guide wheel mounting bracket 423 is configured as the L-shaped block structure, the inner right-angled corner of the first rotary joint guide wheel mounting bracket 423 is mounted at the front right-angled corner of the mounting base 41, the vertically arranged first rotary joint guide wheel 424 is mounted on the outer end surface of the first rotary joint guide wheel mounting bracket 423, the outer right-angled corner of the first rotary joint guide wheel mounting bracket 423 is provided with the horizontally arranged first outer edge, the lower part of the first outer edge is mounted with the horizontally arranged first rotary joint guide wheel 424, the free end of the first pneumatic artificial muscle 421 is connected to the first end of the first rotary joint rotary traction rope 422, the second end of the first rotary joint rotary traction rope 422 sequentially passes around the vertically arranged first rotary joint guide wheel 424, the horizontally arranged first rotary joint guide wheel 424, the first horizontal rope groove and the rope hole at the first end of the swing arm connecting rod 12, and finally connects to the left end and/or the right end of the first rotary joint bearing housing 112. With this configuration, the rotational motion of the first rotary joint 11 is driven by the first rotary joint rotary traction rope 422 passing through the first rotary joint guide wheel 424 mounted on the corresponding first rotary joint guide wheel mounting bracket 423. This motion is controlled by the first pneumatic artificial muscle 421 fixed on both sides of the mounting base 41. The other components and connection relationships are identical to those in Embodiment 1, Embodiment 2 or Embodiment 3.

Specifically, two first rotary joint rotary traction ropes 422 are arranged in a staggered configuration on the rope section above the base adapter module 113.

Detailed description of Embodiment 5: the embodiment is described with reference to FIGS. 1 to 5, according to the present embodiment, the side surface of the second end of the second rotary joint bearing housing 131 is machined into a circular arc surface, and the circular arc surface of the second end of the second rotary joint bearing housing 131 is machined with two second horizontal rope grooves arranged side by side in the upper-lower arrangement;
the second rotary joint rotation and traction mechanism 43 includes two second pneumatic artificial muscles 431, two second rotary joint rotary traction ropes 432, two second rotary joint guide wheel mounting brackets 433 and four second rotary joint guide wheels 434, the two second pneumatic artificial muscles 431 are vertically arranged on left and right sides of the mounting base 41, respectively, the second rotary joint guide wheel mounting bracket 433 is arranged directly above each second pneumatic artificial muscle 431, the second rotary joint guide wheel mounting bracket 433 is configured as the L-shaped block structure, the inner right-angled corner of the second rotary joint guide wheel mounting bracket 433 is mounted at the front right-angled corner of the mounting base 41, the vertically arranged second rotary joint guide wheel 434 is mounted on the outer end surface of the second rotary joint guide wheel mounting bracket 433, the outer right-angled corner of the second rotary joint guide wheel mounting bracket 433 is provided with the horizontally arranged second outer edge, the lower part of the second outer edge is mounted with the horizontally arranged second rotary joint guide wheel 434, the free end of the second pneumatic artificial muscle 431 is connected to the first end of the second rotary joint rotary traction rope 432, the second end of the second rotary joint rotary traction rope 432 sequentially passes around the vertically arranged second rotary joint guide wheel 434, the horizontally arranged second rotary joint guide wheel 434, the second horizontal rope groove at the second end of the second rotary joint bearing housing 131, and finally connects to the left end and/or the right end of the second rotary joint bearing housing 131. With this configuration, the rotational motion of the second rotary joint 13 is driven by the second rotary joint rotary traction rope 432 passing through the second rotary joint guide wheel 434 mounted on the corresponding second rotary joint guide wheel mounting bracket 433. This motion is controlled by the second pneumatic artificial muscle 431 fixed on both sides of the mounting base 41. The other components and connection relationships are identical to those in Embodiment 1, Embodiment 2, Embodiment 3 or Embodiment 4.

Specifically, the two second rotary joint rotary traction ropes 432 are arranged in a staggered configuration on the rope section above the mounting base 41.

Detailed description of Embodiment 6: the embodiment is described with reference to FIGS. 1 to 5, according to the present embodiment, the robot base 4 further includes two base artificial muscle mounting brackets 44, the base artificial muscle mounting bracket 44 is the block structure with the right triangle cross section, the two base artificial muscle mounting brackets 44 are horizontally arranged on left and right sides of the mounting base 41 and arranged above the first pneumatic artificial muscle 421 and the second pneumatic artificial muscle 431, respectively, the inner vertical surface of the base artificial muscle mounting bracket 44 is fixedly connected to the side of the mounting base 41, and the upper horizontal surface of the base artificial muscle mounting bracket 44 is fixedly connected to the lower surfaces of the first pneumatic artificial muscle 421 and the second pneumatic artificial muscle 431. The other components and connection relationships are identical to those in Embodiment 1, Embodiment 2, Embodiment 3, Embodiment 4 or Embodiment 5.

Detailed description of Embodiment 7: the embodiment is described with reference to FIGS. 1 to 5, according to the present embodiment, the four-limb 2R1T parallel mechanism 2 includes the parallel mechanism base 21, two ascending limbs 22, two descending limbs 23, four optical axes 24, four UU mechanisms 25 and eight linear bearings 26; the parallel mechanism base 21 includes the mounting plate connecting column 211, the adapter module connecting plate 212 and two mounting plates 213. The mounting plate connecting column 211 is vertically arranged. Two mounting plates 213 are arranged horizontally on upper and lower sides of the mounting plate connecting column 211. Each mounting plate 213 is machined with four mounting plate mounting holes that penetrate the upper and lower surfaces of the mounting plate and are arranged in a rectangular array. Lower ends of the four optical axes 24 sequentially pass through the mounting plate mounting holes of the two mounting plates 213. The linear shaft 24 is connected to the mounting plate mounting holes through the linear bearing 26 in a sliding manner. The upper ends of the four optical axes 24 are connected to the lower ends of the four UU mechanisms 25, respectively. The upper mounting plate 213 is provided with the adapter module connecting plate 212 adjacent to one side of the dual-joint swing arm 1. The adapter module connecting plate 212 is located above the 2R1T adapter module 132, and the adapter module connecting plate 212 is detachably connected to the 2R1T adapter module 132 through multiple connectors. The upper mounting plate 213 is machined with two ascending limb mounting holes symmetrically arranged on left and right sides centered on the axis of the parallel mechanism base 21. Second ends of the two ascending limbs 22 are vertically inserted into the two ascending limb mounting holes, respectively, and first ends of the two ascending limbs 22 are connected to the upper parts of the linear shaft 24 at two diagonal positions, respectively. The lower mounting plate 213 is machined with two descending limb mounting holes arranged in front and rear positions centered on the axis of the parallel mechanism base 21. Second ends of the descending limbs 23 are vertically inserted into the two descending limb mounting holes, and first ends of the descending limbs 23 are connected to the lower parts of the linear shaft 24 at remaining two diagonals, respectively. With this configuration, the four-limb 2R1T parallel mechanism 2 includes two ascending limbs 22 driven by two lifting pneumatic artificial muscles 221 and two descending limbs 23 driven by two pull-down pneumatic artificial muscles 231, and the ascending limbs 22 and the descending limbs 23 have the same structure. One end of the two lifting pneumatic artificial muscles 221 and the two pull-down pneumatic artificial muscles 231 are fixed with the parallel mechanism base 21, and the other ends of the two lifting pneumatic artificial muscles 221 and the two pull-down pneumatic artificial muscles 231 are respectively fixed with the corresponding optical axes 24 by the lifting artificial muscle free end locking part 222 and the pull-down artificial muscle free end locking part 232, and four UU mechanisms 25 are integrated at the second ends of the four optical axes 24, and the movement of the four optical axes 24 is restricted by the linear bearing 26 on the parallel mechanism base 21. The other components and connection relationships are identical to those in Embodiment 1, Embodiment 2, Embodiment 3, Embodiment 4, Embodiment 5 or Embodiment 6.

Specifically, the artificial muscle fixing end of the descending limb 23 is fixed with the lower bottom surface of the parallel mechanism base 21; the artificial muscle fixing end of the ascending limb 22 is fixed with the upper bottom surface of the parallel mechanism base 21. The artificial muscles in one descending limb 23 and one ascending limb 22 are pressurized and contracted at the same time, and the artificial muscles in the remaining limbs are decompressed and elongated, so that the end traction platform 3 can rotate in one direction along the normal line of the plane formed by the two branches, and the four combinations of the four limbs achieve the rotating motion of the end traction platform 3 around the horizontal axis and the longitudinal axis.

Detailed description of Embodiment 8: the embodiment is described with reference to FIGS. 1 to 5, according to the present embodiment, each ascending limb 22 includes the lifting pneumatic artificial muscle 221 and the lifting artificial muscle free end locking part 222. Two ends of the lifting artificial muscle free end locking part 222 are machined with the lifting artificial muscle free end mounting hole and the upper mounting hole for the linear shaft that penetrate upper and lower surfaces of the locking part, respectively. The free end of the lifting pneumatic artificial muscle 221 is inserted in the lifting artificial muscle free end mounting hole, and the fixed end of the lifting pneumatic artificial muscle 221 is inserted in the ascending limb mounting hole of the parallel mechanism base 21, wherein the optical axes 24 at the two diagonals are inserted in the ascending limb mounting hole. Each descending limb 23 includes the pull-down pneumatic artificial muscle 231 and the pull-down artificial muscle free end locking part 232, both ends of the pull-down artificial muscle free end locking part 232 are machined with the pull-down artificial muscle free end mounting hole and the lower mounting hole for the linear shaft that penetrate the upper and lower surfaces of the locking part, respectively. The free end of the pull-down pneumatic artificial muscle 231 is inserted in the pull-down artificial muscle free end mounting hole, and the fixed end of the pull-down pneumatic artificial muscle 231 is inserted in the descending limb mounting hole of the parallel mechanism base 21, wherein the remaining two diagonal optical axes 24 are inserted in the descending limb mounting hole. The other components and connection relationships are identical to those in Embodiment 1, Embodiment 2, Embodiment 3, Embodiment 4, Embodiment 5, Embodiment 6 or Embodiment 7.

Detailed description of Embodiment 9: the embodiment is described with reference to FIGS. 1 to 5, according to the present embodiment, each UU mechanism 25 includes the first connecting frame 251, the first connecting pin 252, the connecting rod 253, the second connecting pin 254 and the second connecting frame 255. The first connecting frame 251 and the second connecting frame 255 are all U-shaped structures. The U-shaped structure includes the U-shaped frame and the connecting shaft that is vertically arranged and integrated with the middle part of the U-shaped frame web. Two pin holes with the coaxial arrangement are arranged on the two wing plates of the U-shaped frame, and two connecting rod pin holes are arranged at both ends of the connecting rod 253. Both ends of the connecting rod 253 are rotatably connected to the U-shaped frames of the first connecting frame 251 and the second connecting frame 255 by the first connecting pin 252 and the second connecting pin 254, respectively, and the connecting shaft of the second connecting frame 255 is connected to the end traction platform 3. With this configuration, the UU mechanism 25 is configured to connect the end traction platform 3 and the four optical axes 24. When the artificial muscles in one descending limb 23 and one ascending limb 22 are pressurized and contracted at the same time, the artificial muscles in the remaining limbs are decompressed and elongated, the end traction platform 3 can rotate in one direction along the normal line of the plane formed by the two limbs, and the four combinations of the four limbs achieve the rotating motion of the end traction platform 3 around the horizontal axis and the longitudinal axis (spin, and pitch). The other components and connection relationships are identical to those in Embodiment 1, Embodiment 2, Embodiment 3, Embodiment 4, Embodiment 5, Embodiment 6, Embodiment 7 or Embodiment 8.

Detailed description of Embodiment 10: the embodiment is described with reference to FIGS. 1 to 5, according to the present embodiment, the end traction platform 3 includes the traction platform rigid rod member 31, the linear guide 36, two traction pneumatic artificial muscles 32, two wire ropes 33, two guide wheels 34, two guide wheel mounting brackets 35, two sliders 37 and two moving platforms 38. The bottom of the traction platform rigid rod member 31 is mounted with the linear guide 36 in the parallel arrangement, two sliders 37 are slidably mounted on the linear guide 36 in the parallel arrangement, and two symmetrically arranged moving platforms 38 are mounted on bottoms of the two sliders 37, the end surfaces on both sides of the two moving platforms 38 are provided with two horizontal axis holes arranged obliquely and symmetrically, respectively. The axes of the two horizontal axis holes intersect with the middle line of the moving platform 38. Two traction pneumatic artificial muscles 32 are arranged on both sides of the traction platform rigid rod member 31 in a parallel arrangement. Two ear plates are arranged vertically at both ends of the traction platform rigid rod member 31, the two ear plates are located on both sides of the traction platform rigid rod member 31, the fixed ends of the two traction pneumatic artificial muscles 32 are mounted on the corresponding side ear plates, respectively, the free ends of the two traction pneumatic artificial muscles 32 are arranged inward, and the vertically arranged guide wheel 34 is arranged directly in front of the free end of each traction pneumatic artificial muscle 32. The guide wheel 34 is mounted at the end of the traction platform rigid rod member 31 through the guide wheel mounting bracket 35, the free ends of the two traction pneumatic artificial muscles 32 are connected to the first ends of the two wire ropes 33, respectively, and the second ends of the two wire ropes 33 pass around the two guide wheels 34 respectively, and connect to the two moving platforms 38. The four horizontal axis holes on the two moving platforms 38 are inserted with the connecting shaft of the second connecting frame 255 of the four UU mechanisms 25, respectively. With this configuration, the end traction platform 3 is driven by two traction pneumatic artificial muscles 32 arranged on both sides of the traction platform rigid rod member 31 through two steel wire ropes 33 to drive two moving platforms 38 to move along the linear guide 36 by two sliders 37 fixed thereon, and two guide wheels 34 mounted on two guide wheel mounting brackets 35 fixed at both ends of the traction platform rigid rod member 31 play a guiding role for the two steel wire ropes 33, respectively. The other components and connection relationships are identical to those in Embodiment 1, Embodiment 2, Embodiment 3, Embodiment 4, Embodiment 5, Embodiment 6, Embodiment 7, Embodiment 8 or Embodiment 9.

Specifically, the moving platform 38 of the end traction platform 3 is also used as the moving platform of the four-limb 2R1T parallel mechanism 2, and a static platform of the end traction platform 3 is fixed with the broken bone to complete the fracture reduction operation.

### Working Principle

Referring to FIGS. 1 to 5, the working principle of the artificial muscle-driven hybrid fracture reduction surgical robot according to the present disclosure is described as follows: the hybrid fracture reduction surgical robot according to the present disclosure includes two series parts and one parallel part. The serial part of the robot includes the dual-joint swing arm 1 and the end traction platform 3, and the parallel part of the robot includes the four-limb 2R1T parallel mechanism 2. The dual-joint swing arm 1 includes the first rotary joint 11, the swing arm connecting rod 12 and the second rotary joint 13, the first rotary joint 11 is connected to the swing arm connecting rod 12 and the robot base 4, and the second rotary joint 13 is connected to the swing arm connecting rod 12 and the four-limb 2R1T parallel mechanism 2.

From the perspective of robotic mechanism mobility: the dual-joint swing arm 1 enables the robot end a certain range of lateral motion. The end traction platform 3 provides longitudinal motion along the traction direction. The four-limb 2R1T parallel mechanism 2 enables adjustment in the height direction. Collectively, the dual-joint swing arm 1, the four-limb 2R1T parallel mechanism 2, and the end traction platform 3 achieve three-dimensional spatial motion of the mechanism. Specifically, the first rotary joint 11 of the dual-joint swing arm 1 enables rotation motion (yaw) about the vertical axis, while the four-limb 2R1T parallel mechanism 2 provides rotation about the longitudinal axis and the transverse axis (spin, and pitch). The combined motions from these three parts achieve the six-degree-of-freedom movement. This allows the fractured bone end under a certain posture to be pulled for a reduction operation, thereby meeting the demands of reduction surgery.

The above embodiments are merely used for describing the technical solutions of the present disclosure, rather than limiting the same. Although the present disclosure has been described in detail with reference to the preferred examples, those of ordinary skill in the art should understand that the technical solutions of the present disclosure may still be modified or equivalently replaced. However, these modifications or substitutions should not make the modified technical solutions deviate from the spirit and scope of the technical solutions of the present disclosure.

What is claimed is:

1. An artificial muscle-driven hybrid fracture reduction surgical robot, comprising a dual-joint swing arm (1), a four-limb 2R1T parallel mechanism (2), an end traction platform (3) and a robot base (4), wherein the robot base (4) comprises a mounting base (41), two first rotary joint rotation and traction mechanisms (42) and two second rotary joint rotation and traction mechanisms (43), the mounting base (41) is vertically arranged, the first rotary joint rotation and traction mechanism (42) and the second rotary joint rotation and traction mechanism (43) are arranged on left and right sides of the mounting base (41), wherein a top side of the mounting base (41) is provided with the dual-joint swing arm (1), wherein the dual-joint swing arm (1) comprises a first rotary joint (11), a swing arm connecting rod (12) and a second rotary joint (13), wherein the swing arm connecting rod (12) is arranged horizontally, the first rotary joint (11) is mounted at a first end of the swing arm connecting rod (12), left and right ends of the first rotary joint (11) are connected to two first rotary joint rotation and traction mechanisms (42), respectively, a bottom of the first rotary joint (11) is connected to the mounting base (41), a second end of the swing arm connecting rod (12) is mounted with the second rotary joint (13), left and right ends of the second rotary joint (13) are connected to two second rotary joint rotation and traction mechanisms (43), respectively, a top of the second rotary joint (13) is connected to the vertically arranged four-limb 2R1T parallel mechanism (2), and a top end of the four-limb 2R1T parallel mechanism (2) is mounted with an end traction platform (3); the four-limb 2R1T parallel mechanism (2) comprises a parallel mechanism base (21), two ascending limbs (22), two descending limbs (23), four optical axes (24), four UU mechanisms (25) and eight linear bearings (26); the parallel mechanism base (21) comprises a mounting plate connecting column (211), an adapter module connecting plate (212) and two mounting plates (213), the mounting plate connecting column (211) is vertically arranged, two mounting plates (213) are arranged horizontally on upper and lower sides of the mounting plate connecting column (211), each mounting plate (213) is machined with four mounting plate mounting holes that penetrate upper and lower surfaces of the mounting plate and are arranged in a rectangular array, lower ends of the four optical axes (24) sequentially pass through the mounting plate mounting holes of the two mounting plates (213), the linear shaft (24) is connected to the mounting plate mounting holes through the linear bearing (26) in a sliding manner, upper ends of the four optical axes (24) are connected to lower ends of the four UU mechanisms (25), respectively, the upper mounting plate (213) is provided with the adapter module connecting plate (212) adjacent to a side of the dual-joint swing arm (1), the adapter module connecting plate (212) is located above the 2R1T adapter module (132), and the adapter module connecting plate (212) is detachably connected to the 2R1T adapter module (132) through a plurality of connectors, the upper mounting plate (213) is machined with two ascending limb mounting holes symmetrically arranged on left and right sides centered on an axis of the parallel mechanism base (21), second ends of the two ascending limbs (22) are vertically inserted into the two ascending limb mounting holes, respectively, and first ends of the two ascending limbs (22) are connected to upper parts of the linear shaft (24) at two diagonal positions, respectively; wherein the lower mounting plate (213) is machined with two descending limb mounting holes arranged in front and rear positions centered on an axis of the parallel mechanism base (21), wherein second ends of the descending limbs (23) are vertically inserted into the two descending limb mounting holes, and first ends of the descending limbs (23) are connected to lower parts of the linear shaft (24) at remaining two diagonals, respectively.

2. The artificial muscle-driven hybrid fracture reduction surgical robot according to claim 1, wherein the first rotary joint (11) comprises a first rotary joint bearing (111), a first rotary joint bearing housing (112) and a base adapter module (113), wherein a first rotary joint mounting hole vertically penetrating upper and lower surfaces of the swing arm connecting rod (12) is machined at a first end of the swing arm connecting rod (12), the first rotary joint bearing

(111) is embedded inside the first rotary joint mounting hole, an upper end of the first rotary joint bearing housing (112) is inserted into an inner hole of the first rotary joint bearing (111), and a lower end of the first rotary joint bearing housing (112) is connected to the base adapter module (113).

3. The artificial muscle-driven hybrid fracture reduction surgical robot according to claim 2, wherein the second rotary joint (13) comprises a second rotary joint bearing, a second rotary joint bearing housing (131) and a 2R1T adapter module (132), a second rotary joint mounting hole vertically penetrating upper and lower surfaces of the swing arm connecting rod is machined at a second end of the swing arm connecting rod (12), the second rotary joint bearing is embedded inside the second rotary joint mounting hole, a lower end of the second rotary joint bearing housing (131) is inserted into an inner hole of the second rotary joint bearing, and an upper end of the second rotary joint bearing housing (131) is connected to the 2R1T adapter module (132).

4. The artificial muscle-driven hybrid fracture reduction surgical robot according to claim 3, wherein one end of the swing arm connecting rod (12) adjacent to the robot base (4) is machined into a circular arc surface, and the circular arc surface is machined with two first horizontal rope grooves arranged side by side in an upper-lower arrangement, and two rope holes penetrating an inner side surface of the first rotary joint mounting hole are machined at a first end of the swing arm connecting rod (12), and the two rope holes correspond to the two first horizontal rope grooves one-to-one respectively;
wherein the first rotary joint rotation and traction mechanism (42) comprises two first pneumatic artificial muscles (421), two first rotary joint rotary traction ropes (422), two first rotary joint guide wheel mounting brackets (423) and four first rotary joint guide wheels (424), the two first pneumatic artificial muscles (421) are vertically arranged on left and right sides of the mounting base (41), respectively, the first rotary joint guide wheel mounting bracket (423) is arranged directly above each first pneumatic artificial muscle (421), wherein the first rotary joint guide wheel mounting bracket (423) is configured as an L-shaped block structure, an inner right-angled corner of the first rotary joint guide wheel mounting bracket (423) is mounted at a front right-angled corner of the mounting base (41), the vertically arranged first rotary joint guide wheel (424) is mounted on an outer end surface of the first rotary joint guide wheel mounting bracket (423), an outer right-angled corner of the first rotary joint guide wheel mounting bracket (423) is provided with a horizontally arranged first outer edge, a lower part of the first outer edge is mounted with the horizontally arranged first rotary joint guide wheel (424), a free end of the first pneumatic artificial muscle (421) is connected to a first end of the first rotary joint rotary traction rope (422), a second end of the first rotary joint rotary traction rope (422) sequentially passes around the vertically arranged first rotary joint guide wheel (424), the horizontally arranged first rotary joint guide wheel (424), the first horizontal rope groove and the rope hole at the first end of the swing arm connecting rod (12), and finally connects to a left end and a right end of the first rotary joint bearing housing (112).

5. The artificial muscle-driven hybrid fracture reduction surgical robot according to claim 4, wherein a side surface of a second end of the second rotary joint bearing housing (131) is machined into a circular arc surface, and the circular arc surface of the second end of the second rotary joint bearing housing (131) is machined with two second horizontal rope grooves arranged side by side in an upper-lower arrangement;
wherein the second rotary joint rotation and traction mechanism (43) comprises two second pneumatic artificial muscles (431), two second rotary joint rotary traction ropes (432), two second rotary joint guide wheel mounting brackets (433) and four second rotary joint guide wheels (434), wherein the two second pneumatic artificial muscles (431) are vertically arranged on left and right sides of the mounting base (41), respectively, the second rotary joint guide wheel mounting bracket (433) is arranged directly above each second pneumatic artificial muscle (431), the second rotary joint guide wheel mounting bracket (433) is configured as an L-shaped block structure, an inner right-angled corner of the second rotary joint guide wheel mounting bracket (433) is mounted at a front right-angled corner of the mounting base (41), the vertically arranged second rotary joint guide wheel (434) is mounted on an outer end surface of the second rotary joint guide wheel mounting bracket (433), an outer right-angled corner of the second rotary joint guide wheel mounting bracket (433) is provided with a horizontally arranged second outer edge, a lower part of the second outer edge is mounted with the horizontally arranged second rotary joint guide wheel (434), a free end of the second pneumatic artificial muscle (431) is connected to a first end of the second rotary joint rotary traction rope (432), a second end of the second rotary joint rotary traction rope (432) sequentially passes around the vertically arranged second rotary joint guide wheel (434), the horizontally arranged second rotary joint guide wheel (434), the second horizontal rope groove at a second end of the second rotary joint bearing housing (131), and finally connects to a left end and a right end of the second rotary joint bearing housing (131).

6. The artificial muscle-driven hybrid fracture reduction surgical robot according to claim 5, wherein the robot base (4) further comprises two base artificial muscle mounting brackets (44), the base artificial muscle mounting bracket (44) is a block structure with a right triangle cross section, wherein the two base artificial muscle mounting brackets (44) are horizontally arranged on left and right sides of the mounting base (41) and arranged above the first pneumatic artificial muscle (421) and the second pneumatic artificial muscle (431) respectively, wherein an inner vertical surface of the base artificial muscle mounting bracket (44) is fixedly connected to a side of the mounting base (41), and an upper horizontal surface of the base artificial muscle mounting bracket (44) is fixedly connected to lower surfaces of the first pneumatic artificial muscle (421) and the second pneumatic artificial muscle (431).

7. The artificial muscle-driven hybrid fracture reduction surgical robot according to claim 6, wherein each ascending limb (22) comprises a lifting pneumatic artificial muscle (221) and a lifting artificial muscle free end locking part (222), two ends of the lifting artificial muscle free end locking part (222) are machined with a lifting artificial muscle free end mounting hole and an upper mounting hole for the linear shaft that penetrate upper and lower surfaces of the locking part, respectively, wherein a free end of the lifting pneumatic artificial muscle (221) is inserted in the lifting artificial muscle free end mounting hole, and a fixed end of the lifting pneumatic artificial muscle (221) is inserted in the ascending limb mounting hole of the parallel mechanism base (21), wherein the optical axes (24) at the two diagonals are inserted in the ascending limb mounting hole; wherein each descending limb (23) comprises a pull-down pneumatic artificial muscle (231) and a pull-down artificial muscle free end locking part (232), both ends of the pull-down artificial muscle free end locking part (232) are machined with a pull-down artificial muscle free end mounting hole and a lower mounting hole for the linear shaft that penetrate the upper and lower surfaces of the locking part, respectively, a free end of the pull-down pneumatic artificial muscle (231) is inserted in the pull-down artificial muscle free end mounting hole, and a fixed end of the pull-down pneumatic artificial muscle (231) is inserted in the descending limb mounting hole of the parallel mechanism base (21), wherein the remaining two diagonal optical axes (24) are inserted in the descending limb mounting hole.

8. The artificial muscle-driven hybrid fracture reduction surgical robot according to claim 7, wherein each UU mechanism (25) comprises a first connecting frame (251), a first connecting pin (252), a connecting rod (253), a second connecting pin (254) and a second connecting frame (255), the first connecting frame (251) and the second connecting frame (255) are all U-shaped structures, wherein the U-shaped structure comprises a U-shaped frame and a connecting shaft that is vertically arranged and integrated with a middle part of the U-shaped frame web, two pin holes with a coaxial arrangement are arranged on the two wing plates of the U-shaped frame, and two connecting rod pin holes are arranged at both ends of the connecting rod (253), both ends of the connecting rod (253) are rotatably connected to the U-shaped frames of the first connecting frame (251) and the second connecting frame (255) by the first connecting pin (252) and the second connecting pin (254), respectively, and a connecting shaft of the second connecting frame (255) is connected to the end traction platform (3).

9. The artificial muscle-driven hybrid fracture reduction surgical robot according to claim 8, wherein the end traction platform (3) comprises a traction platform rigid rod member (31), a linear guide (36), two traction pneumatic artificial muscles (32), two wire ropes (33), two guide wheels (34), two guide wheel mounting brackets (35), two sliders (37) and two moving platforms (38), wherein a bottom of the traction platform rigid rod member (31) is mounted with the linear guide (36) in a parallel arrangement, two sliders (37) are slidably mounted on the linear guide (36) in a parallel arrangement, and two symmetrically arranged moving platforms (38) are mounted on bottoms of the two sliders (37), end surfaces on both sides of the two moving platforms (38) are provided with two horizontal axis holes arranged obliquely and symmetrically, respectively, wherein the axes of the two horizontal axis holes intersect with a middle line of the moving platform (38), two traction pneumatic artificial muscles (32) are arranged on both sides of the traction platform rigid rod member (31) in a parallel arrangement, two ear plates are arranged vertically at both ends of the traction platform rigid rod member (31), the two ear plates are located on both sides of the traction platform rigid rod member (31), fixed ends of the two traction pneumatic artificial muscles (32) are mounted on the corresponding side ear plates, respectively, wherein free ends of the two traction pneumatic artificial muscles (32) are arranged inward, and a vertically arranged guide wheel (34) is arranged directly in front of a free end of each traction pneumatic artificial muscle (32), the guide wheel (34) is mounted at an end of the traction platform rigid rod member (31) through the guide wheel mounting bracket (35), free ends of the two traction pneumatic artificial muscles (32) are connected to first ends of the two wire ropes (33), respectively, and second ends of the two wire ropes (33) pass around the two guide wheels (34) respectively, and connect to the two moving platforms (38), and the four horizontal axis holes on the two moving platforms (38) are inserted with the connecting shaft of the second connecting frame (255) of the four UU mechanisms (25), respectively.
